(19) United States
(12) Patent Application Publication
(10) Pub. No.: US 2010/0191137 A1
(43) Pub. Date: Jul. 29, 2010

(54) DEVICE FOR DETECTING DIAPHRAGM MOVEMENTS

(75) Inventors: Jiri BRADA, Praha (CZ); Pavel SMRCKA, Praha (CZ); Karel HANA, Svetice - Ricany u Prahy (CZ); Jan KASPAR, Jicin (CZ); Radek FIALA, Chotebor (CZ); Martin VITEZNIK, Praha (CZ); Jozef ROSINA, Praha (CZ); Peter KNEPPO, Bratislava (SK); Jan MUZIK, Praha (CZ)

Correspondence Address: WRB-IP LLP, 801 N. Pitt Sreet , Suite 123, ALEXANDRIA, VA, 22314, US

(73) Assignees: Ceske vysoke uceni technicke v Praze, Fakulta biomedicinskeho inzenyrstvi (Czech Technical; University, Kladno (CZ)

(21) Appl. No.: 12/692,684

(22) Filed: Jan. 25, 2010

(30) Foreign Application Priority Data
Jan. 23, 2009 (CZ) PUV 2009-20780

(51) Int. Cl. A61B 5/08

(52) U.S. Cl. 600/534

(57) ABSTRACT

A device for detecting diaphragm movements includes an accelerometric sensor with a fixture for attachment to the user's chest and a conductor connected to the control module. The device is useful for detecting diaphragm movements during invasive curative procedures on the human heart, in particular during operations of atrial fibrillation.

### TECHNICAL FIELD

The technical solution concerns a device for detecting diaphragm movements during invasive curative procedures on the human heart, in particular during operations of atrial fibrillation.

### STATE OF THE ART

Ablation technology has recently undergone a rapid development in cardiac electrophysiology. Some of this technology has been already used commonly, while other technology is being clinically tested at present. These systems are used for a more effective curative treatment of cardiac arrhythmia. More commonly used systems include radio frequency systems, cryoablation systems, laser ablation systems and high-intensity focused ultrasound ablation systems. They are used most of all during operations of fibrillation of the left atrium. During this surgical intervention, the operating surgeons are trying to reach transmural, continuous lesions using these systems and to interrupt abnormal re-entry circuits around the entries of pulmonary veins, i.e. to achieve electrical isolation of pulmonary veins.

The phrenic nerve, nervus phrenicus, is a mixed nerve with a prevalence of motor fibres. Its right and left branches originate from the cervical plexus, influence numerous organs and pass through the diaphragm, which they innervate. The anatomical structures of the phrenic nerve pass in the proximity of mostly right cardiac pulmonary veins. During the operation as mentioned above, this nerve could be partly or fully destroyed, in particular if a balloon catheter is used. This results in highly unfavourable impacts on the patient as regards the function of his respiratory system. At present, it is very difficult to detect the continuity of the function of the phrenic nerve in real time during interventions.

The intracardial stimulation of the phrenic nerve by means of an introduced electrophysiological catheter intended to provide stimulation in adequate places leads to the activation of the muscular parts of the diaphragm. This muscular stimulation of the diaphragm results in a visible movement of the chest, similar to hiccups. In practice, this type of stimulation is commonly used during operations; chest movements are then detected to verify the correct function of the phrenic nerve using the operating surgeon's hand laid on the chest. No other method is known as yet.

Theoretically, the function of the respiratory muscle can be detected using an oesophageal catheter with a helix electrode, reading the electromyographical signal from the adjacent structures inside the oesophagus. However, its practical use for detecting any potential damage to the phrenic nerve seems to be too complicated and costly, and therefore this method is not used.

To ensure detection of phrenic nerve stimulation, some stimulation systems of implantable stimulators and defibrillators apply the functions of a lymphatic sensor indicating respiratory activity. This is, however, an undesirable side effect of stimulation and the stimulation system tries to eliminate this type of stimulation. Taking into account the need of implantation and a highly specific target use of detection, these systems cannot be used for our specific case.

### ESSENCE OF THE TECHNICAL SOLUTION

The above-mentioned issue of real-time detection of the function of the phrenic nerve is solved by a device for detecting diaphragm movements. This device facilitates the monitoring of these movements, allowing a quicker and more reliable response by the operating surgeon during intracardial ablation therapy.

The main functional part of the device is an accelerometric sensor, attached to the user's chest. It is a standard precise double-axis low-power accelerometer based on iMEMS, i.e. integrated Micro Electrical Mechanical System. Its output signal is an analogue signal proportional to its acceleration. The signal from this sensor is transmitted via a cable to a module that integrates a supply system, a DC-DC converter, an impedance section and simple filters. The analogue output from the module corresponding with diaphragm movements, i.e. chest movements, is used for connection to any EP system for arrhythmia mapping, e.g. CardioLab EP System Prucka, EP Tracer, etc. Conductors with standard connectors are designed for connecting the module and the EP system.

In the control SW of the EP system used, it is necessary to define the inputs to which the module is connected, to set their amplification and, if applicable, filtration, and to determine the location of the signal progress on the screen.

During the operation and in case of application of ablation energy, the operating surgeon has the possibility, after general connection and setting, to monitor the diaphragm movement curve on the EP system screens, along with stimulation signals, the intracardial signal, etc. The operating surgeon can monitor the correlation of these movements with the stimulation spikes of the signals generated by stimulation catheters. If he identifies a loss of diaphragm movements from the signal on the screen during the ongoing phrenic nerve stimulation, the phrenic nerve is likely to have been interrupted. This is an indicator for immediate and quick interruption of ablation therapy.

Essential advantages of this device for detecting diaphragm movements are its low acquisition costs and its overall simplicity. After the initial setting with the EP system used in the operating theatre, it requires no operation other than to attach the sensor to the specific patient. A big undisputed advantage is that the device is absolutely non-invasive; the accelerometric sensor is only attached to the user's chest mechanically, on a standard adhesive ECG electrode. The device does not have any conductive connection to the user.

### LIST OF DRAWINGS

The device for detecting diaphragm movements as described in this technical solution will be described in detail on a specific embodiment using the attached drawings,

where FIG. 1 shows the connection diagram of the device for detecting diaphragm movements to the EP system and to the patient and

FIG. 2 shows a block diagram of the module for detecting diaphragm movements.

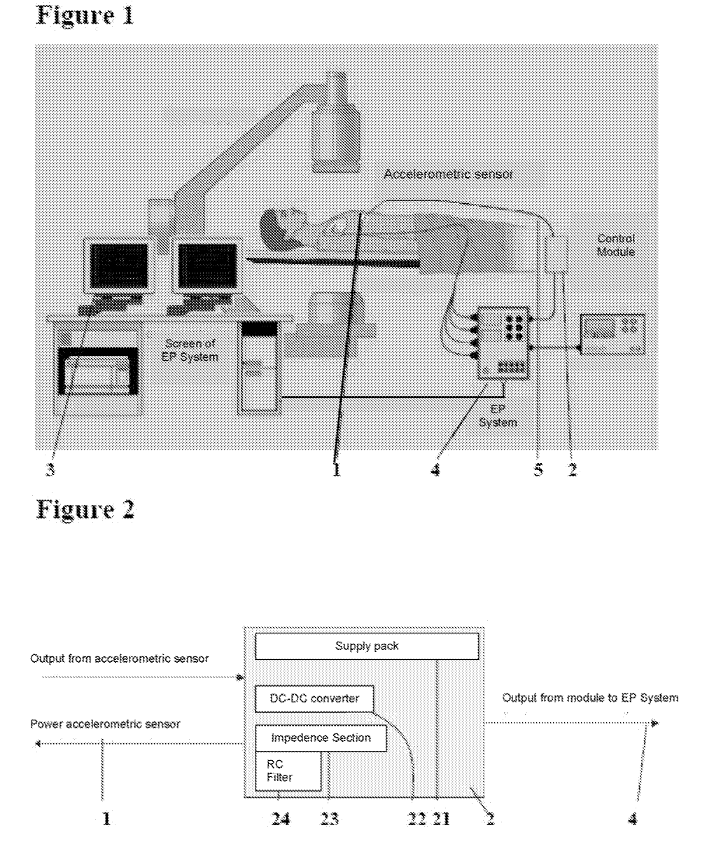

### EMBODIMENTS OF THE TECHNICAL SOLUTION

As shown in FIG. 1, the device for detecting diaphragm movements concerned is composed of an accelerometric sensor 1, which is connected to the control module 2 via conductor 5.

The accelerometric sensor 1 is lodged in a light polyurethane casing. The low weight of the casing does not burden the accelerometric sensor 1 to avoid any potential added errors. From one side, a press button is attached to the casing of the sensor 1 for mechanical attachment to the adhesive ECG electrode. This electrode is attached to the required place in the chest area The accelerometer is an iMEMS-based sensor—integrated Micro Electrical Mechanical System. The sensor is oriented in the casing to ensure that its output shows the maximum amplitude of the output voltage during chest movements. The single-axis sensor is supplied via a conductor 5 from the control module 2. This conductor 5 also transmits the analogue output signal from the accelerometric sensor 1, which is proportional to the chest deviation. Its output level does not exceed 0.7 to 4.2V, at the typical sensitivity of 1.7V per g.

The control module 2 itself comprises a supply pack 21 for the accelerometric sensor 1—two AAA batteries, a DC-DC converter 22 5V and a fundamental impedance section 23, which contains a simple RC filter 24. The casing is made from plastic and contains a supply switch and two control diodes. One indicates the ON-OFF state of the module and the other signals a low voltage on the inserted AAA batteries. The module casing contains an input connector for connecting the conductor 5 from the accelerometric sensor 1 and output connectors for output conductors that transmit the signal to the EP system 4, which is not part of the device.

These output conductors and their connectors comply with the DIN 42802 standard. The length of the conductor 5 between the accelerometric sensor 1 and the control module 2 is approximately 2 m. The reason is the installation of the control module 2 in the bottom part of the operation table or in its proximity, close to the input connectors to the EP system 4, connected to the screen 3.

### INDUSTRIAL USE

The device for detecting diaphragm movements as specified in this invention can be used in medicine, especially during invasive curative procedures on the human heart, in particular during operations of atrial fibrillation.

1. A device for detecting diaphragm movements, comprising an accelerometric sensor with a fixture for attachment to the user's chest and a conductor connected to the control module.

2. The device as in claim 1, wherein the accelerometric sensor comprises a double-axis low-power accelerometer.

3. The device as in claim 2, wherein the control module contains a supply pack, a DC-DC converter, an impedance part and simple filters.

4. The device as in claim 3, wherein the control module is attached to a screen displaying the diaphragm movement curve.

4. The device as in claim 2, wherein the control module is attached to a screen displaying the diaphragm movement curve.

5. The device as in claim 1, wherein the control module contains a supply pack, a DC-DC converter, an impedance part and simple filters.

6. The device as in claim 1, wherein the control module is attached to a screen displaying the diaphragm movement curve.
